(19) United States
(12) Patent Application Publication
(10) Pub. No.: US 2003/0185402 A1
(43) Pub. Date: Oct. 2, 2003

(54) Adaptive distortion manager for use with an acoustic echo canceler and a method of operation thereof

(75) Inventors: Jacob Benesty, Summit, NJ (US); Tomas F. Gaensler, Chatham, NJ (US)

Correspondence Address: HITT GAINES P.C., P.O. BOX 832570, RICHARDSON, TX, 75083, US

(73) Assignee: Lucent Technologies, Inc., Murray Hill, NJ

(21) Appl. No.: 10/107,223

(22) Filed: Mar. 27, 2002

(51) Int. Cl. H04B 3/20; A61F 11/06; G10K 11/16; H03B 29/00; H04M 9/08

(52) U.S. Cl. 381/66; 379/406.08; 381/71.11

(57) ABSTRACT

A distortion manager and a method of managing distortion for use with an acoustic echo canceler. In one embodiment, the distortion manager includes a coherence ascertainer coupled to an adaptive distortion adder. The coherence ascertainer determines a coherency between audio streams and the adaptive distortion adder selectively adds non-linear distortion to at least one of the audio streams based on the coherency.

### TECHNICAL FIELD OF THE INVENTION

The present invention is directed, in general, to acoustic echo cancelling systems and, more specifically, to a distortion manager for use with an acoustic echo canceler, a method of managing distortion associated with an acoustic echo canceler and an acoustic echo canceler employing the same.

### BACKGROUND OF THE INVENTION

Teleconferencing is now widely used to conduct business. Many existing teleconferencing systems, which range from simple speaker-phones to modern video teleconferencing equipment, have a single full-duplex audio channel for voice communication. These monophonic systems typically employ acoustic echo cancelers to remove undesired echos that result from acoustic coupling. Typically, an acoustic echo canceler employs an adaptive filter to estimate the impulse response from the loudspeaker to the microphone in a room in which an echo occurs and generates a signal to electrically cancel that echo.

In teleconferencing, the acoustic coupling results when sound emitted from a teleconference loudspeaker, which is in response to a signal from a remote location, arrives at a teleconference microphone. The microphone generates a signal, for example an echo, in response to this sound. The generated microphone signal is then transmitted to the remote location. If nothing were done to cancel the acoustic echo signal, the echo would continue to circulate between the teleconferencing locations producing undesirable multiple echoes.

Like monophonic teleconferencing, high-quality stereophonic teleconferencing also requires acoustic echo cancelling. Stereophonic acoustic echo cancelling, however, presents a problem which does not exist in the monophonic context. Unlike monophonic acoustic echo cancelers, conventional stereophonic acoustic echo cancelers do not independently estimate the individual impulse responses of a room. Rather, conventional stereophonic acoustic echo canceler systems derive impulse responses which have a combined effect of reducing echo. The problem with deriving impulse response estimates based on the combined effect of reduced echo is that such combined effect does not necessarily mean that the actual individual impulse responses are accurately estimated. Unless individual impulse responses are accurately estimated, the ability of the acoustic echo canceler system to be robust to changes in the acoustic characteristics of the remote location is limited and undesirable lapses in performance may occur.

Accurately estimating individual impulse responses of a two-channel echo cancellation system needs special attention because of its inherent non-uniqueness problem. For example, if multiple channel signals, such as two in a teleconferencing system, originate from the same source, there is no unique echo path solution for the echo canceler to identify. One way to mitigate this non-uniqueness problem is to diminish the linear relation between the channel signals or, in other words, decorrelate the channel signals. This decorrelation must of course be done carefully in a way that the stereo effect is not degraded, and the introduced distortion is essentially inaudible.

A successful method for decorrelating the channel signals may be achieved by introducing a small non-linearity into each channel to reduce the interchannel coherence. Preferably, the amount of non-linear distortion added to each or either channel signal is small to preserve the perceptual quality of the channel signals. One method of adding static nonlinearity to decorrelate channel signals is proposed in U.S. Pat. No. 5,828,756 to Benesty, et al. (“Benesty”), entitled “Stereophonic Acoustic Echo Cancellation Using Non-Linear Transformations,” issued Oct. 27, 1998 and incorporated herein by reference.

The method proposed in Benesty has been proven not to destroy the stereo effect of the channel signals, and for speech signals is virtually inaudible when a minimum amount of nonlinear distortion is added. For high quality speech (i.e., 8 kHz bandwidth) and music, even a minimum amount of added nonlinear distortion may be objectionable. This may be due to the fact that rectifiers boost higher frequencies which become audible due to poor masking from the original speech at these frequencies.

Ideally, the channel signals would pass uncorrelated audio streams without distorting them to preserve a high audio quality in the receiving room. When the signals originate from the same source, i.e., when they are linearly related or highly coherent, some distortion may need to be introduced to avoid the problem of non-uniqueness for the echo canceler. There may be no need for decorrelation, however, when multiple talkers are active or when there is background music playing since the normal equation to be solved by the echo canceler in this case is indeed nonsingular.

Accordingly, what is needed in the art is a way to accurately estimate individual impulse responses in acoustic echo cancelers by adding non-linear distortion only when needed to reduce correlation.

### SUMMARY OF THE INVENTION

To address the above-discussed deficiencies of the prior art, the present invention provides a distortion manager for use with an acoustic echo canceler. In one embodiment, the distortion manager includes a coherence ascertainer coupled to an adaptive distortion adder. The coherence ascertainer is configured to determine a coherency between audio streams and the adaptive distortion adder is configured to selectively add non-linear distortion to at least one of the audio streams based on the coherency.

In another aspect, the present invention provides a method of managing distortion associated with an acoustic echo canceler. The method includes determining a coherence between audio streams and adding non-linear distortion selectively to at least one of the audio streams based on the coherence.

The present invention also provides, in yet another aspect, an acoustic echo canceler for a stereophonic teleconferencing system. The acoustic echo canceler includes an echo estimator, an echo error determiner and a distortion manager. The echo estimator produces a total echo estimate of individual echo paths in a receiving location of the stereophonic teleconferencing system by filtering audio streams from a transmitting location of the stereophonic teleconferencing system based on estimated impulse responses of the receiving location. The echo error determiner generates a signal representing the difference between the total echo estimate and a signal at the receiving location representing at least acoustic echo signals. The distortion manager includes a coherence ascertainer and an adaptive distortion adder. The coherence ascertainer determines a coherency between the audio streams and the adaptive distortion adder, which is coupled to the coherence ascertainer, and selectively adds non-linear distortion to at least one of the audio streams based on the coherency.

The foregoing has outlined preferred and alternative features of the present invention so that those skilled in the art may better understand the detailed description of the invention that follows. Additional features of the invention will be described hereinafter that form the subject of the claims of the invention. Those skilled in the art should appreciate that they can readily use the disclosed conception and specific embodiment as a basis for designing or modifying other structures for carrying out the same purposes of the present invention. Those skilled in the art should also realize that such equivalent constructions do not depart from the spirit and scope of the invention.

### BRIEF DESCRIPTION OF THE DRAWINGS

For a more complete understanding of the present invention, reference is now made to the following descriptions taken in conjunction with the accompanying drawings, in which:

FIG. 1 illustrates a system diagram of an embodiment of a stereophonic teleconferencing system employing an acoustic echo canceler constructed in accordance with the principals of the present invention;

FIG. 2 illustrates a block diagram of an embodiment of a distortion manager constructed in accordance with the principles of the present invention;

FIG. 3 illustrates a flow diagram of an embodiment of a method of managing distortion associated with an acoustic echo canceler, constructed in accordance with the principles of the present invention;

FIG. 4 illustrates an echo path response used to simulate an acoustic path in accordance with the principles of the present invention;

FIG. 5a illustrates an estimated magnitude-squared coherence for measured speech signals with small regularization of the algorithm in Table 1 in accordance with the principles of the present invention;

FIG. 5b illustrates an estimate d magnitude-squared coherence for measured speech signals with normal regularization of the algorithm in Table 1 in accordance with the principles of the present invention;

FIG. 6 illustrates a level of added non-linear distortion as a function of Equation 27 in accordance with the principles of the present invention; and

FIG. 7 illustrates the performance of an adaptive distortion manager constructed in accordance with the principles of the present invention.

### DETAILED DESCRIPTION

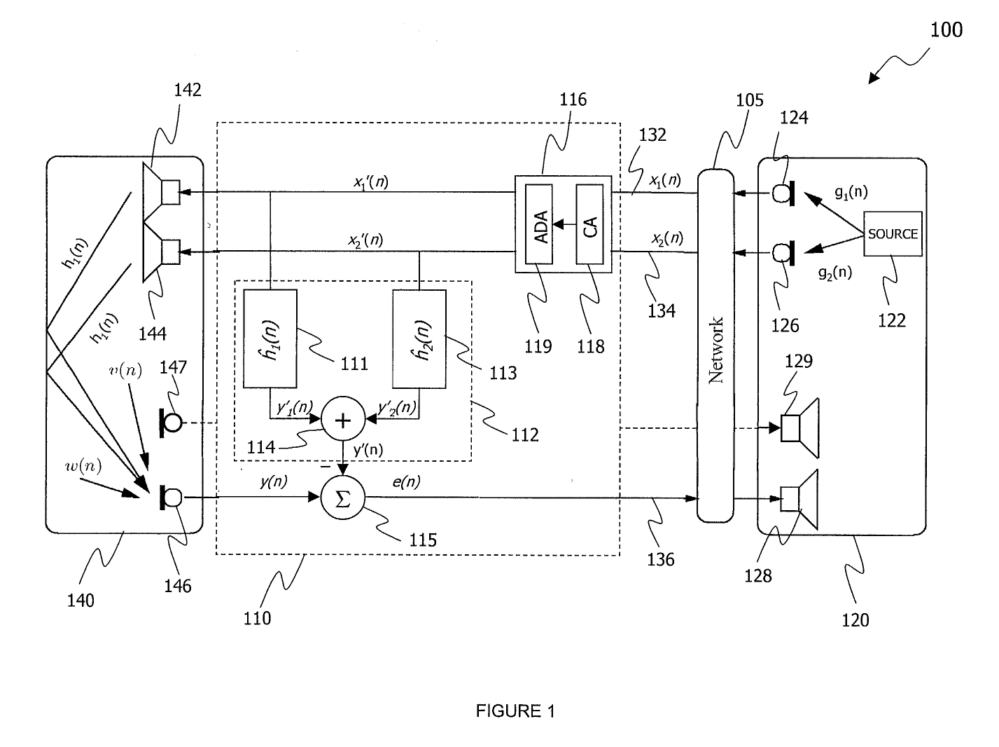

Referring initially to FIG. 1, illustrated is a system diagram of an embodiment of a stereophonic teleconferencing system, generally designated 100, employing an acoustic echo canceler 110 constructed in accordance with the principals of the present invention. The stereophonic teleconferencing system 100 is employable with a network 105 and includes components placed at a transmitting location (e.g., transmission room) 120 and a receiving location (e.g., receiving room) 140. The acoustic echo canceler 110 includes an echo estimator 112, an echo error determiner 115 and a distortion manager 116. The echo estimator 112 includes a first filter 111, a second filter 113 and an adder 114. The distortion manager 116 includes a coherence ascertainer 118 and an adaptive distortion adder 119.

The transmission room 120 includes an acoustic source 122, a first microphone 124, a second microphone 126, a first return loudspeaker 128 and a second return loudspeaker 129. The network 105 includes a first path 132, a second path 134, and a return path 136. The receiving room 140 includes a first loudspeaker 142, a second loudspeaker 144, a first return microphone 146 and a second return microphone 147.

Except for the acoustic echo canceler 110, the stereophonic teleconferencing system 100 is a conventional two-channel teleconferencing system. In the transmission room 120, the first microphone 124 and the second microphone 126 detect and receive signals from the acoustic source 122 via two acoustic paths that are characterized by the impulse responses g₁(n) and g₂(n). Typically, the acoustic source 122 is a person in the transmission room 120 who is speaking to another person or persons in the receiving room 140. In the illustrated embodiment, it is assumed that the acoustic paths include the responses from the first return loudspeaker 128, the second return loudspeaker 129, the first microphone 124 and the second microphone 126. The outputs from the first microphone 124 and the second microphone 126 are stereophonic audio streams x₁(n), x₂(n), respectively.

The first return loudspeaker 128 and the second return loudspeaker 129 receive audio streams from the receiving room 140 via the network 105. In the illustrated embodiment, the second loudspeaker 129 and the second return microphone 147 are not coupled by a second return path of the network 105 in order to simplify the discussion. One skilled in the art will understand that the discussion with respect to the first return path 136 also applies to a second return path.

The stereophonic audio streams, x₁(n), x₂(n), are transmitted from the first microphone 124 and the second microphone 126 through the acoustic echo canceler 110 via the network 105 to the first loudspeaker 142 and the second loudspeaker 144 in the receiving room 140. The distortion manager 116 of the acoustic echo canceler 110 receives the audio streams x₁(n), x₂(n) from the network 105. The coherence ascertainer 118 of the distortion manager 116 determines the coherence between the audio streams x₁(n), x₂(n). The adaptive distortion adder 119, coupled to the coherence ascertainer 118, selectively adds non-linear distortion to audio streams x₁(n) and x₂(n) based on a coherency level γ determined by the coherence ascertainer 118. The non-linear distortion may be added to either one or both of the audio streams x₁(n), x₂(n).

The coherency level γ is a measure of the linear correlation between the two stereophonic audio streams x₁(n), x₁(n) One skilled in the art will understand that the coherence level γ between the audio streams x₁(n), x₂(n) is equal to one when the audio streams x₁(n), x₂(n) are linearly dependent. The addition of non-linear distortion to one or both of the audio streams x₁(n), x₂(n) decorrelates the audio streams x₁(n), x₂(n) and reduces the coherence level γ to some value below one. The audible degradation of the audio streams x₁(n), x₂(n) created by the addition of non-linear distortion to each audio stream x₁(n), x₂(n) can be minimized by adding non-linear distortion that is a corresponding signal of each audio stream x₁(n), x₂(n). The audible degradation can be further reduced by only adding the non-linear distortion when the coherence level γ is about 1, such as greater than 0.9. Additionally, the audible degradation can be reduced by only adding a minimum amount of non-linear distortion to sufficiently decorrelate the audio streams x₁(n), x₂(n). A factor α may be used to quantify the level of introduced non-linear distortion.

In one embodiment, a maximum level of non-linear distortion α added is about 0.5. In another embodiment, non-linear distortion α is not added when the coherency level γ is less than about 0.9. In other embodiments, the non-linear distortion α is added when the coherency level γ is greater than about 0.9. In still other embodiments, varying amounts of non-linear distortion α are added based on the coherency level γ. For example, if the coherency level γ is about 0.95, then the adaptive distortion adder 119 may add about 0.25 of the non-linear distortion α. On the other hand, if the coherency level γ is about 0.975, then the adaptive distortion adder 119 may add 0.4 of the non-linear distortion α. One skilled in the art will understand that varying amounts of non-linear distortion α may be added based on the coherency level γ.

The addition of non-linear distortion α transforms the audio streams x₁(n), x₂(n) into processed audio streams x₁′(n), x₂′(n). In the illustrated embodiment, the designator “′” indicates a transformed audio stream, such as x₁′(n), which advantageously has a reduced correlation with the other transformed audio stream of the stereophonic system, such as x₂′(n). In the echo estimator 112, the transformed audio streams x₁′(n), x₂′(n) are used to derive an estimate of the echo in the receiving room 140 by driving the first filter 111 and the second filter 113.

In the illustrated embodiment, the first filter 111 and the second filter 113 are finite impulse response (FIR) filters with adjustable coefficients that model acoustic impulse responses h₁(n), h₂(n), of the echo path in the receiving room 140. The coefficients of the first filter 111 and the second filter 113 may be derived using conventional techniques, such as a stochastic gradient algorithm. Though preferably located in the receiving room 140, the first filter 111 and the second filter 113 may be located anywhere in the system, such as the transmission room 120 or other locations within the network 105.

Driven by the transformed audio streams x₁′(n), x₂′(n) the first filter 111 and the second filter 113 produce signals y₁′(n), y₂′(n) which are added together by the adder 114 to produce a total echo estimate y′(n) as the output of the echo estimator 112. The output of the echo estimator 112, y′(n), is subtracted from a receiving room signal y(n) by the echo error determiner 115 to produce an error signal e(n). The error signal e(n) is intended to be small (i.e., driven towards zero) in the absence of near-end speech (i.e., speech generated in the receiving room 140). The coefficients of the first filter 111 and the second filter 113 are updated in an effort to reduce the error signal e(n) to zero. The error signal e(n) is then transmitted by the acoustic echo canceler 110 across the first return path 136 of the network 105 to the first return speaker 128 in the transmission room 120.

The network 105 is typically a conventional telecommunications network that may be either wireless, hardwired or a combination of the two. The network 105 is used to couple the transmission room 120 to the receiving room 140. Typically, the receiving room 140 is remotely located from the transmission room 120.

In the receiving room 140, the first loudspeaker 142 and the second loudspeaker 144 are acoustically coupled to the first return microphone 146 in the receiving room 140 via the paths indicated by impulse responses h₁(n), h₂(n). The output of the first return microphone 146 is the receiving room signal y(n) which represents acoustic signals in the receiving room 140 being detected and received by the first return microphone 146. Generally, the receiving room signal y(n) is composed of an echo yₑ(n), ambient noise w(n) and possibly receiving room speech v(n), which is typically referred to as double-talk. Thus the receiving room signal 140 model is represented by:

y(n)=yₑ(n)+v(n)+w(n),

where yₑ(n)=Σₚ₌₁²hₚ(n)*xₚ(n) is the echo, * denotes convolution and h₁(n), h₂(n), are the acoustic impulse responses of the receiving room 140 echo paths.

As is common in a two-channel system, the first loudspeaker 142 and the second loudspeaker 144 are also acoustically coupled to the second return microphone 148 by other acoustic paths. Typically, four adaptive filters, therefore, are needed for a conventional stereophonic system. In the illustrated embodiment, only two adaptive filters, the first filter 111 and the second filter 113, are shown in order to simplify the discussion of the acoustic echo canceler 110. As discussed above with respect to the second return loudspeaker 129, only the acoustic coupling to the first return microphone 146 will be discussed. One of ordinary skill in the art will understand that the analysis concerning the acoustic echo canceler 110 for the output of the first return microphone 146 is applicable to the output of the second return microphone 147 as well. Similarly, one skilled in the art will also understand that the acoustic echo canceler 110 may function for the outputs of the first microphone 124 and the second microphone 126 in the transmission room 120 as discussed with respect to the first return microphone 146 and the second return microphone 147 in the receiving room 140. In this respect, the functions of the receiving room 140 and the transmission room 120 are exchanged.

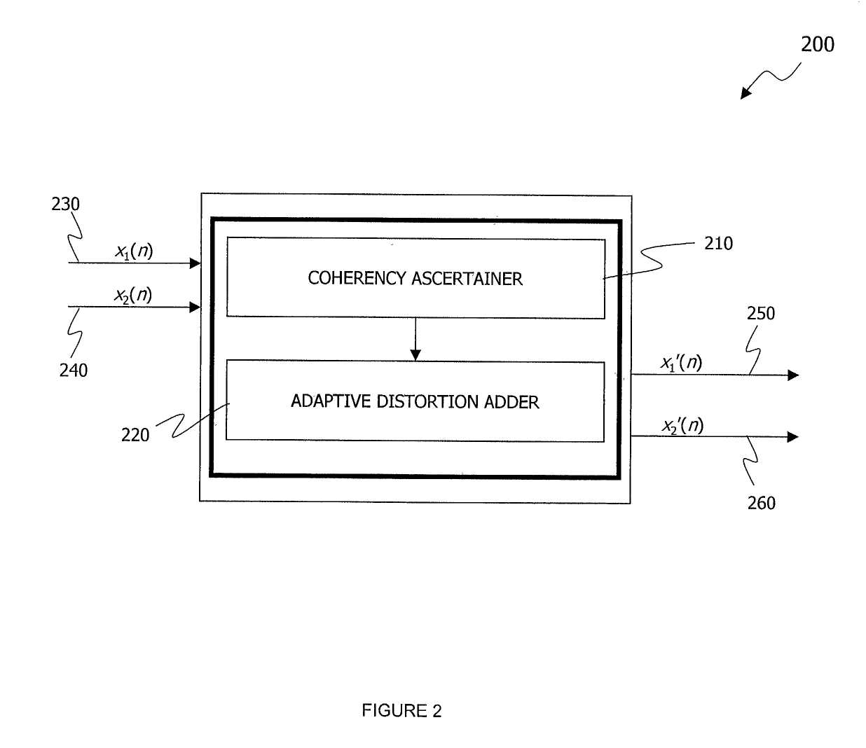

Turning now to FIG. 2, illustrated is a block diagram of an embodiment of a distortion manager, generally designated 200, constructed in accordance with the principles of the present invention. The distortion manager 200 includes a coherence ascertainer 210 and an adaptive distortion adder 220, and is coupled to a first input path 230, a second input path 240, a first output path 250 and a second output path 260.

The coherence ascertainer 210 determines the coherence of the audio streams x₁(n), x₂(n) on the first input path 230 and the second input path 240, and sends a coherence level γ to the adaptive distortion adder 220. The coherence level between processed audio streams x₁′(n), x₂′(n), denoted by γ_{α}, is discussed in “Investigation of Several Types of Non-linearities For Use In Stereo Acoustic Echo Cancellation,” by D. R. Morgan, et al., IEEE Trans. Speech Audio Processing, vol. 9, September 2001, which is incorporated herein by reference. As demonstrated below, the coherence level γ_{α}, may be used to determine the coherence level γ between audio streams x₁(n), x₂(n).

The coherence γ_{α} is given by Equation (1) as a function of the spectra and cross-spectra of x₁, x₂, \tilde{x}₁ and \tilde{x}₂ for transmission signals x₁, x₂.

$${γ}_{α} ( f ) = \frac{{S}_{{x}_{1} {x}_{2}} ( f ) + β {S}_{{\overline{x}}_{1} {\overline{x}}_{2}} ( f )}{{{[ {S}_{{x}_{1} {x}_{1}} ( f ) + β {S}_{{\overline{x}}_{1} {\overline{x}}_{1}} ( f ) ]}^{1 / 2} [ {S}_{{x}_{2} {x}_{2}} ( f ) + β {S}_{{\overline{x}}_{2} {\overline{x}}_{2}} ( f ) ]}^{1 / 2}} , \qquad ( 1 )$$

whereβ is a constant depending on the nonlinear function employed (for example, a half-wave rectifier) and S_{xpxq}(f), p, q =1, 2 are the cross-spectra and auto-spectra of the corresponding transmission signals x₁, x₂. The spectra can be computed from the corresponding cross correlation functions r_{xpxq}(1) according to Equation (2)

$${S}_{{x}_{p} {x}_{q}} ( f ) = {\sum }_{l = - ∞}^{∞} {T}_{{x}_{p} {x}_{q}} ( l ) {ⅇ}^{- j2π fl} . \qquad ( 2 )$$

For a positive and negative half-wave rectifier β is defined by Equation (3) as

$$β = \frac{{α}^{2}}{1 + α} . \qquad ( 3 )$$

Further, the transmission signals x₁, x₂, are modeled as constant spectrum (white) Gaussian signals. The coherence between the transmission signals x₁, x₂, is also constant γ≧0, and the transmission signals x₁, x₂, are band-limited in frequency between ±fₛ/2 with variance σₓ² where fₛ denotes a sampling frequency. After sampling, an anechoic model is represented by Equation (4a) and Equation (4b)

Tₓ_{ₚ}ₓ_{ₚ}(l)=E{xₚ(n)xₚ(n−l)}=σₓ²δ(l), p=1,2,−x<l<x, (4a)

Tₓ_{₁}ₓ_{₂}(l)=E{x₁(n)x₂(n−l)=γσₓ²δ(l−l₀),−∞<l<x, (4b)

where δ(l) is the unity impulse function and l the time lag variable, and lₒ is a possible time shift between channels. Applying Equation (2) to the anechoic model results in Equation (5a), Equation (5b) and Equation (6)

Sₓ_{ₚ}ₓ_{ₚ}(f)=rₓ_{ₚ}ₓ_{ₚ}(0)=σₓ_{¹}²p=1,2,∀f, (5a)

Sₓ_{₁}ₓ_{₂}(f)=rₓ_{₁}ₓ_{₂}(l _{O})=γσₓ²e^{-j2πjl}^{₀},∀f., (5a)

$$γ = \vert {γ}_{x1x2} ( f ) \vert = \frac{\vert {S}_{{x}_{1} {x}_{2}} ( f ) \vert}{\sqrt{{S}_{{x}_{1} {x}_{1}} ( f ) {S}_{{x}_{2} {x}_{2}} ( f )}} \cdot ∀ f . \qquad ( 6 )$$

The magnitude, therefore, of the coherence between the channels before passing the nonlinearity which in this example is a positive and negative half-wave-rectifier, is constant and equal to γy(≧0) for this model. Computing the spectra S\tilde{x}ₚ, S\tilde{x}_{q}(f), p,q=1, 2 is somewhat more complicated. Expressions for r\tilde{x}ₚ\tilde{x}_{q}(l), p,q=1, 2 can be found as a function of rxₚx_{q}(l), p,q=1, 2 by using the methods outlined in “The Correlation Function of Gaussian Noise Passed Through Nonlinear Devices,” by R. F. Baum, IEEE Trans. Inform. Theory, vol. IT-15, July 1969 and incorporated herein by reference. The expressions for auto correlation of the signals \tilde{x}₁,\tilde{x}₂ is given in Equation (7)

$${r}_{{\overline{x}}_{p} {\overline{x}}_{p}} ( l ) = \frac{{σ}_{x}^{2}}{2 π} \{ {ρ}_{{x}_{p} {x}_{p}} ( l ) {cos}^{- 1} [ - {ρ}_{{x}_{p} {x}_{p}} ( l ) ] + \sqrt{1 - {ρ}_{{x}_{p} {x}_{p}}^{2} ( l )} \} , p = 1.2 . , \qquad ( 7 )$$

where ρₓₚₓₚ(l) is the normalized correlation function given in Equation (8)

$${ρ}_{{x}_{p} {x}_{p}} ( l ) = \frac{{r}_{{x}_{p} {x}_{p}} ( l )}{{σ}_{x}^{2}} . \qquad ( 8 )$$

The normalized cross-correlation function ρₓ₁ₓ₂(l) is analogously defined.

The sign difference between a positive and a negative half-wave rectifier disappears in the autocorrelation function. The cross-correlation between a positive half of a signal (\tilde{x}₊) and a negative half (\tilde{x}₋), however, needs special attention. The simplest method of finding this function is to observe Equation (9)

ρ_{\tilde{x}}_{₊}_{\tilde{x}}_{₋}(l)=ρ_{\tilde{x}}_{₊}_{\tilde{e}}(l)−ρ_{\tilde{x}}_{₊}_{\tilde{x}}_{₊}(l). (9)

Using Equation (7) and the following Equation (10)

$${ρ}_{\overline{x} \vert x} ( l ) = \frac{1}{2} {ρ}_{xx} ( l ) \cdot ( Gaussian signals ) , \qquad ( 10 )$$

yields Equation (11)

$${r}_{{\overline{x}}_{1} {\overline{x}}_{2}} ( l ) = \frac{{σ}_{x}^{2}}{2} {ρ}_{{x}_{1} {x}_{2}} ( l ) - \frac{{σ}_{x}^{2}}{2 π} \{ {ρ}_{{x}_{1} {x}_{2}} ( l ) {cos}^{- 1} [ - {ρ}_{{x}_{1} {x}_{2}} ( l ) ] + \sqrt{1 - {ρ}_{{x}_{1} {x}_{2}}^{2} ( l )} \} . \qquad ( 11 )$$

The corresponding spectra of Equation (7) and Equation (11) is then shown in Equation (12a) and Equation (12b)

$$\begin{matrix}{S}_{{\overline{x}}_{p} {\overline{x}}_{p}} ( f ) = \frac{{σ}_{x}^{2}}{2} \{ 1 + \frac{1}{π} [ δ ( f ) - 1 ] \} \cdot p = 1.2 , & ( 12 a ) \\ {S}_{{\overline{x}}_{1} {\overline{x}}_{2}} ( f ) = \frac{{σ}_{x}^{2}}{2} {γⅇ}^{- j2π {fl}_{0}} - \frac{{σ}_{x}^{2}}{2 \overline{n}} [ {γcos}^{- 1} ( - γ ) + \sqrt{1 - {γ}^{2}} - 1 + δ ( f ) ] {ⅇ}^{- j2π {fl}_{0}} . & ( 12 b )\end{matrix}$$

Combining Equation (1) with Equations (12a), (12b) results in Equation (13)

$$\vert {γ}_{α} ( f ) = \frac{{γσ}_{x}^{2} + {βσ}_{x}^{2} \{ \frac{γ}{2} - \frac{1}{2 π} \{ {γcos}^{- 1} ( - γ ) + \sqrt{1 - {γ}^{2}} - 1 + δ ( f ) ] \}}{{σ}_{x}^{2} + β \frac{{σ}_{x}^{2}}{2} \{ 1 + \frac{1}{π} [ δ ( f ) - 1 ] \}} \cdot ∀ f , . \qquad ( 13 )$$

Furthermore, when f≠0, Equation (13) results in Equation (14) which can be rewritten as Equation (15) and Equation (16) with Equation (17) representing the closed form of F_{γ}⁻¹ and Equation (18) representing a simple recursion of F_{γ}⁻¹.

$$\begin{matrix}{γ}_{α} = \vert {γ}_{α} ( f ) \\ = \frac{γ + \frac{β}{2} \{ γ - \frac{1}{π} [ γ {cos}^{- 1} ( - γ ) + \sqrt{1 - {γ}^{2}} - 1 ] \}}{1 + \frac{β}{2} ( 1 - \frac{1}{π} )} \\ = F ( α , γ ) , f \neq 0 ,\end{matrix} \qquad ( 14 )$$

α=F_{α}⁻¹(γ,γ_{α}), (15)

γ=F_{γ}⁻¹(α,γ_{α}), (16)

$$\begin{matrix}α = \frac{{γ}_{α} - γ + \sqrt{( {γ}_{α} - γ ) \{ γ - {γ}_{α} - \frac{2}{π} [ γ cos \frac{1}{π} ( - γ ) + \sqrt{1 - {γ}^{2}} - 1 - {γ}_{α} ] \}}}{γ - {γ}_{α} ( 1 - \frac{1}{π} ) - \frac{1}{π} [ γ {cos}^{- 1} ( - γ ) - \sqrt{1 - {γ}^{2}} - 1 ]} , & ( 17 ) \\ \begin{matrix}γ ( n ) = - \frac{β}{2} \{ γ ( n - 1 ) - \frac{1}{π} [ γ ( n - 1 ) {cos}^{- 1} [ - γ ( n - 1 ) ] + \\ \sqrt{1 - {γ}^{2} ( n - 1 )} - 1 ] \} + \\ [ 1 + \frac{β}{2} ( 1 - \frac{1}{π} ) ] {γ}_{α} .\end{matrix} & ( 18 ) \\ γ ( 0 ) = {γ}_{α} . & \end{matrix}$$

Additionally, a simple closed form expression for misalignment is given for a two channel frequency domain algorithm in Equation (19)

$$\frac{E \{ {‖ ϵ ( m ) ‖}^{2} \}}{{‖ h ‖}^{2}} = \frac{( 1 - λ )}{2} \frac{{σ}_{b}^{2}}{{‖ h ‖}^{2}} tr \{ {S}^{- 1} \} . \qquad ( 19 )$$

From Equation (19), Equation (20) can be written expressing the excess misalignment (“ex. mis”) as solely dependent on channel coherence.

$${J}_{ex . mis} = \frac{l}{L} {\sum }_{l = 0}^{L - 1} \frac{1}{1 - {\vert γ ( l ) \vert}^{2}} \geq 1 , \qquad ( 20 )$$

where L represents the length of the adaptive filter and l=0 . . . L−1 represents the frequency bin numbers.

In addition to the above equations, the coherence ascertainer 210 may determine the coherence of the audio streams x₁(n), x₂(n) by employing an adaptive algorithm to obtain an estimate of the coherence with very low computational complexity. The adaptive algorithm may be a two-channel frequency-domain algorithm that computes the magnitude coherence explicitly in order to update the estimate of the echo path.

In one embodiment, the coherence ascertainer 210 may calculate an estimate coherence level \hat{γ} employing Equation (21) of the two-channel frequency-domain adaptive algorithm for echo cancellation given in Table 1. For each iteration, this algorithm uses a block of L samples to update the estimated echo path ĥₚ(m),p=1,2, with ĥₚ defined by Equation (22).

$${\hat{\underline{h}}}_{p} = {F}_{2 L \times 2 L} [ \begin{matrix}{\hat{h}}_{p} \\ {0}_{L \times 1}\end{matrix} ] , \qquad ( 22 )$$

where ĥₚ is a modeling filter. Using Equation (21) from the

TABLE 1

| | |
|---|---|
| Definitions | |
| $\begin{matrix}{G}_{2 L \times 2 L}^{01} - {F}_{2 L \times 2 L} [ \begin{matrix}{0}_{L \times L} & {0}_{L \times L} \\ {0}_{L \times L} & {I}_{L \times L}\end{matrix} ] {F}_{2 L \times 2 L}^{- 1} \\ {G}_{2 L \times 2 L}^{10} - {F}_{2 L \times 2 L} [ \begin{matrix}{I}_{L \times L} & {0}_{L \times L} \\ {0}_{L \times L} & {0}_{L \times L}\end{matrix} ] {F}_{2 L \times 2 L}^{- 1} \\ G = {G}_{2 L \times 2 L}^{10} , constrained algorithm \\ G - {I}_{2 L \times 2 L} / 2 , unconstrained algorithm \\ {μ}^{′} - μ ( 1 - λ ) , 0 \leq 1 μ \leq 1\end{matrix}$ | |
| Spectral estimation | |
| $\begin{matrix}{D}_{p} ( m ) = diag \{ {{F}_{2 L \times 2 L} [ {x}_{p} ( mL - L ) … {x}_{p} ( mL + L - 1 ) ]}^{T} \} \cdot p = 1.2 \\ {\tilde{S}}_{{x}_{p} {x}_{q}} ( m ) - λ {\tilde{S}}_{{x}_{p} {x}_{q}} ( m - 1 ) + ( 1 - λ ) {D}_{p}^{*} ( m ) {D}_{q} ( m ) , p , q - 1 , 2 \\ {\tilde{S}}_{{x}_{p} {x}_{q}} ( m ) = {\tilde{S}}_{{x}_{p} {x}_{p}} ( m ) + diag \{ {δ}_{p .0} … {δ}_{p .2 L - 1} \} , p = 1 , 2\end{matrix}$ | (21) |
| $\begin{matrix}{\vert Γ ( m ) \vert}^{2} = {[ {\tilde{S}}_{{x}_{1} {x}_{1}} ( m ) {\tilde{S}}_{{x}_{2} {x}_{2}} ( m ) ]}^{- 1} {\tilde{S}}_{{x}_{2} {x}_{1}} ( m ) {\tilde{S}}_{{x}_{1} {x}_{2}} ( m ) \\ {S}_{p} ( m ) = {\tilde{S}}_{{x}_{p} {x}_{p}} ( m ) [ {I}_{2 L \times 2 L} - {\vert Γ ( m ) \vert}^{2} ] , p , q = 1 , 2 \\ {K}_{1} ( m ) = {S}_{1}^{- 1} ( m ) [ {D}_{1}^{*} ( m ) - {\tilde{S}}_{{x}_{1} {x}_{2}} ( m ) {\tilde{S}}_{{x}_{2} {x}_{2}}^{- 1} ( m ) {D}_{2}^{*} ( m ) ] \\ {K}_{2} ( m ) = {S}_{2}^{- 1} ( m ) [ {D}_{2}^{*} ( m ) - {\tilde{S}}_{{x}_{2} {x}_{1}} ( m ) {\tilde{S}}_{{x}_{1} {x}_{1}}^{- 1} ( m ) {D}_{1}^{*} ( m ) ]\end{matrix}$ | |
| Echo canceler | |
| $\begin{matrix}\underline{e} ( m ) = \underline{y} ( m ) = {G}_{2 L \times 2 L}^{01} [ {D}_{1} ( m ) {\hat{\underline{h}}}_{1} ( m - 1 ) + {D}_{2} ( m ) {\hat{\underline{h}}}_{2} ( m - 1 ) ] \\ {\hat{\underline{h}}}_{p} ( m ) - {\hat{\underline{h}}}_{p} ( m - 1 ) + 2 {μ}^{′} {GK}_{p} ( m ) \underline{e} ( m ) \cdot p - 1 , 2 \end{matrix}$ | |

frequency-domain algorithm in Table 1, the magnitude squared coherence for the processed audio streams x₁′(n), x₂′(n), is estimated by Equation (23) where \tilde{γ}_{α}(l,m) is the estimated coherence at frequency f=1/2L for time block m.

|Γ(m)|²=diag{|\tilde{γ}_{α}(0,m)|²|\tilde{γ}_{α}(1, m)² . . . |\tilde{γ}_{α}(2L−1,m)|²}. (23)

Assuming that the coherence is constant with frequency, then the excess misalignment is kept below a certain desired level J_{ex. mis, d} as reflected in Equation (24)

$${J}_{ex . mis} \leq {J}_{ex . mis , d} = \frac{l}{L} {\sum }_{l = 0}^{L - 1} \frac{1}{1 - {\vert {γ}_{α , d} ( l ) \vert}^{2}} = \frac{1}{1 - {\vert {γ}_{α , d} ( l ) \vert}^{2}} . \qquad ( 24 )$$

The desired magnitude coherence can then be given by Equation (25)

$${\vert {γ}_{α , d} \vert}^{2} = 1 - \frac{1}{{J}_{ex . mis , d}} . \qquad ( 25 )$$

Using Equation (20) and the main diagonal of Equation (23), an estimation of the excess misalignment may be given by Equation (26)

$${\hat{J}}_{ex . mis} ( m ) = \frac{1}{L} {\sum }_{l = 0}^{L - 1} \frac{1}{1 - {\vert {\hat{γ}}_{α} ( l , m ) \vert}^{2}} . \qquad ( 26 )$$

From Equation (26), an average magnitude coherence can be calculated that results in an equivalent amount of excess misalignment as represented by Equation (26)

$${\vert {\hat{γ}}_{α} ( m ) \vert}^{2} = 1 - \frac{1}{{\hat{J}}_{ex . mis} ( m )} . \qquad ( 26 )$$

Using Equation (16) and the above equations, therefore, the coherence ascertainer 210 may calculate an estimate coherence level \hat{γ} from the following Equation (27)

\hat{γ}(m)=F_{γ}⁻¹{\hat{α}(m)₁\hat{γ}_{α}(m)}, (27)

where m represents a time block, \hat{α} is the estimated non-linearity level and \hat{γ}_{α} is the estimated coherency level of processed audio streams x₁′(n), x₂′(n).

The adaptive distortion adder 220 adds non-linear distortion α to the input audio streams x₁(n), x₂(n) based on the coherency level γ determined by the coherence ascertainer 210. In some embodiments, non-linear distortion α may be added to only one of the input audio streams x₁(n), x₂(n). In one embodiment, the adaptive distortion adder 220 may add non-linear distortion α by applying a non-linear transformation module to the input audio streams x₁(n), x₂(n), as discussed in the incorporated reference. The addition of the non-linear distortion α will transform the input audio streams x₁(n), x₂(n) into processed audio streams x₁′(n), x₂′(n), and ensure that the coherence magnitude between the processed audio streams x₁′(n), x₂′(n) will be smaller than one. The processed audio streams x₁′(n), x₂′(n) exit the distortion manager 200 on the first output path 250 and the second output path 260, respectively.

In a preferred embodiment, the adaptive distortion adder 220 may add the non-linear distortion α to the audio streams x₁(n), x₂(n) based on Equation (27). After an estimate of the coherence level \hat{γ} between the audio streams x₁(n), x₂(n) is obtained by employing Equation (27), non-linear distortion α may be added to at least one of the input audio streams x₁(n), x₂(n) based on Equation (28) in order to obtain the desired coherency.

\hat{α}ₜₑₘₚ=F_{α}⁻¹{\hat{γ}(m),γ_{α,d}}, (28)

wherein \hat{α}ₜₑₘₚ represents an estimate of the temporary non-linear distortion α and γ_{a,d} represents the desired coherence level. Essentially, the non-linear distortion α is applied to the next block of data \hat{α}(m+1) resulting in \tilde{γ}≦γ_{α,d}. The estimate may be bound according to Equation (29) in order to preserve the perceived quality of the audio streams x₁(n), x₂(n).

\hat{α}(m+1)=min{αₘₐₓ, max(\hat{ₜₑₘₚ},0)}. (29 )

In some embodiments, the maximum level of the non-linear distortion α may be about 0.5.

In one embodiment, the adaptive distortion adder 220 may employ a half-wave rectifier function. In other embodiments, the adaptive distortion adder 220 may employ any other non-linear function such as, for example, a full-wave rectifier function, a hard limiter function, a square-law function, a square-sign function, a cubic function or any of a number of other non-linear functions which will be both obvious and familiar to one of ordinary skill in the art.

An example of a distortion manager 200 may be illustrated using the coherence estimate of the algorithm in Table 1 and the equations of Table 2. In this example, real-life speech is used as described in P. Eneroth et al., Acoustic signal Processing for Telecommunications, (S. L. Gay and J. Benesty eds., Kluwer Academic Publishers, 2000) and incorporated herein by reference. The source in the transmission room is a stereo recording with a male talker. At times 30.9, 61.8, 66.9, 72.1 and 77.2 seconds, there are talker position changes. Additionally, from 40 seconds to 50 seconds there is some background music playing which is somewhat shifted in the stereo image plane toward the left channel.

TABLE 2

Initialization and Definitions
$\begin{matrix}\hat{α} ( 0 ) = {α}_{max} \\ {x}_{p} ( m ) = {[ {x}_{p} ( mL ) {…x}_{p} ( mL + L - 1 ) ]}^{T} , p = 1 , 2\end{matrix}$
Design Specifications
$\begin{matrix}{x}_{1}^{′} ( m ) = {x}_{1} ( m ) + \frac{\hat{α} ( m )}{2} [ {x}_{1} ( m ) + \vert {x}_{1} ( m ) \vert ] \\ {x}_{2}^{′} ( m ) = {x}_{1} ( m ) + \frac{\hat{α} ( m )}{2} [ {x}_{1} ( m ) + \vert {x}_{1} ( m ) \vert ]\end{matrix}$
$\begin{matrix}{\hat{J}}_{ex . mis} ( m ) = \frac{1}{L} {\sum }_{l = 0}^{L - 1} \frac{1}{1 - {\vert \tilde{γ} α ( l , m ) \vert}^{2}} \\ \hat{γ} α ( m ) = \sqrt{1 - \frac{1}{{\hat{J}}_{ex . mis} ( m )}}\end{matrix}$
$\begin{matrix}\tilde{γ} ( m ) = {F}_{γ}^{- 1} [ \hat{α} ( m ) , {\dot{γ}}_{α} ( m ) ] \\ {\hat{α}}_{temp} = {F}_{α}^{1} [ \hat{γ} ( m ) , {γ}_{a , d} ] \\ \tilde{α} ( m + 1 ) = min [ {α}_{max} \cdot max ( {\hat{α}}_{temp} \cdot 0 ) ]\end{matrix}$

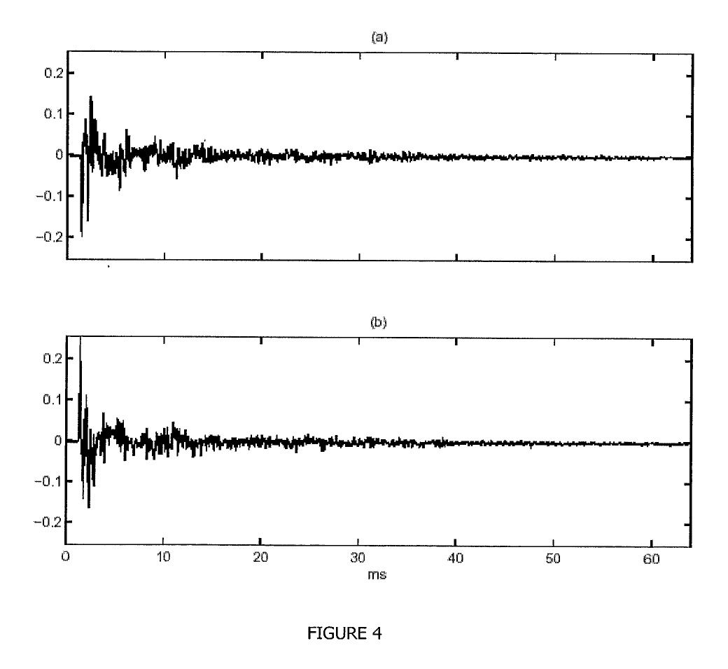

The receiving room speech is generated by filtering the (nonlinearly) processed transmission room speech through an echo path model. This model is a measured acoustic response between a left loudspeaker and a standard cardioid microphone positioned on top of a workstation. The original impulse response has a length of 256 ms, consisting of 4096 coefficients at 16 kHz sampling rate. In this simulation, however, the echo path is restricted to 1024 coefficients as illustrated in FIG. 4. The ambient noise level is ENR=σ_{ye}²/σ_{w}²≈1000 (30 dB)and the adaptive filter parameters are

L=1024 (64 ms), λ=[1−1/(3·2L)]^{L}, μ=1, ĥ(0)=0.

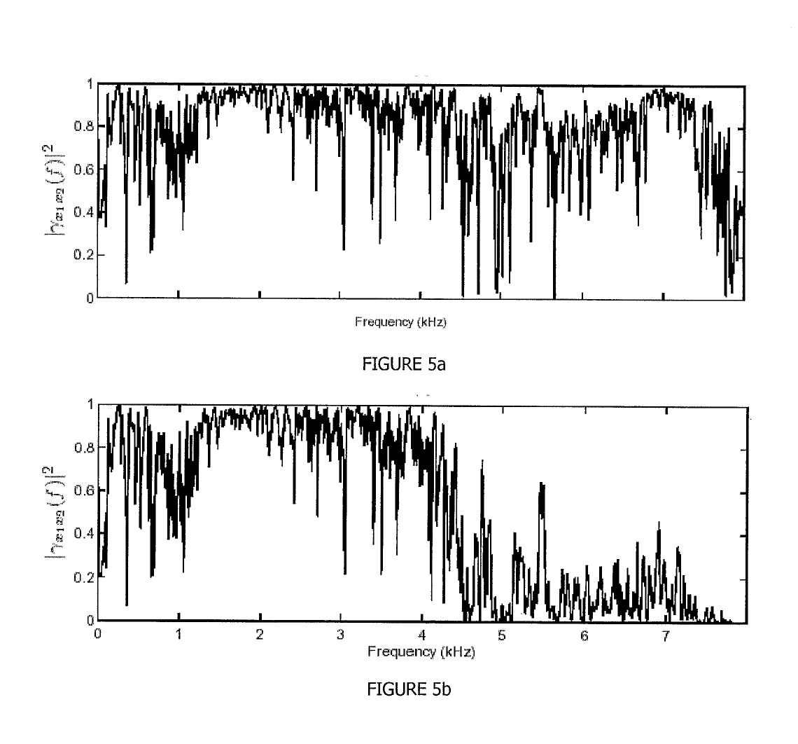

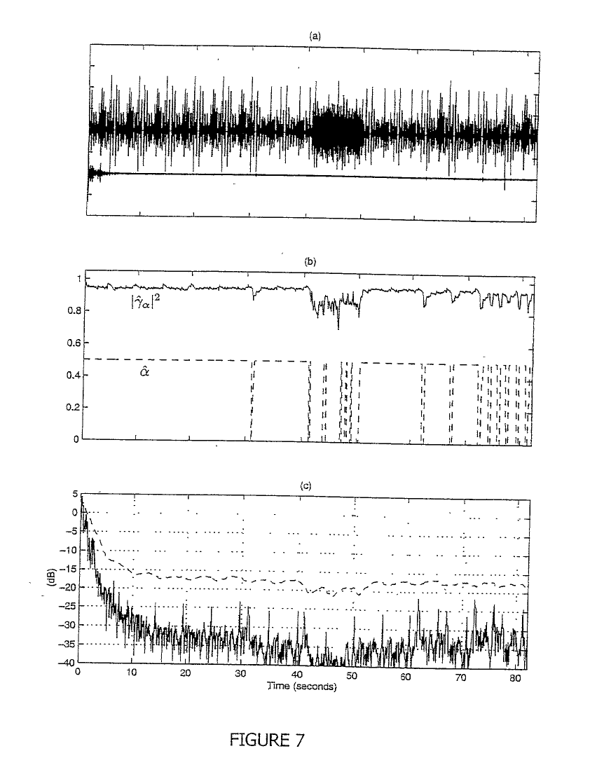

Additionally, δ=5σₓ² as shown in FIGS. 5a and 7 and δ=5·10⁻⁵σₓ² as shown in FIG. 5b.

First, the magnitude-squared coherence of the above described transmission room speech as a function of frequency is studied. These estimates, regularized (δ(·,·)>0) and unregularized (δ(·,·)=0), given by Equation (21) are shown in FIGS. 5a, 5b, respectively. These estimates were obtained when there is no talker position change or background music. Not surprisingly, the regularization severely biases the coherence estimate at higher frequencies where the speech level is lower. It is therefore advantageous to use only lower frequencies when averaging the squared coherence function of Equation (26), and, accordingly, the estimates will be modified so that only coherence values over the interval 1=L/8+1 . . . L/2, i.e., 1000 to 4000 Hz are used.

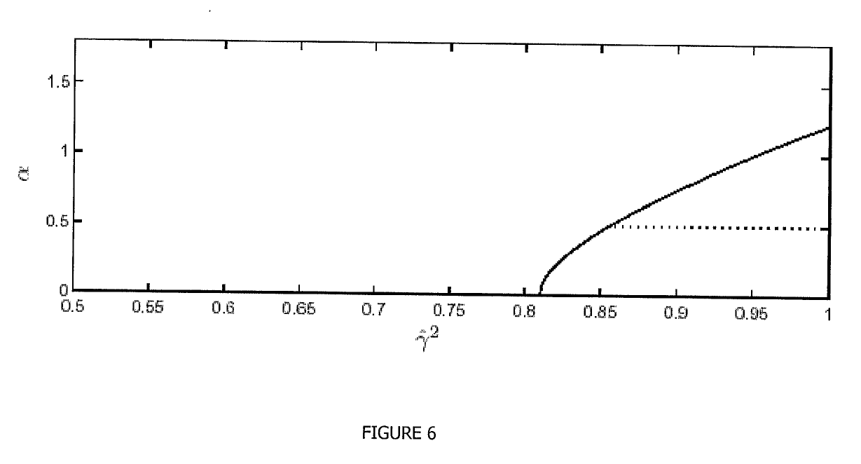

Table 2 shows the whole algorithm for the adaptive nonlinearity that is used in this simulation. FIG. 6 illustrates the applied non-linear distortion α as a function of magnitude coherence of the unprocessed transmission signals or audio streams x₁(n), x₂(n). In FIG. 6, the desired processed coherence γ_{α,d} was chosen to be 0.9. The solid line in the figure presents the level of nonlinearity. The dashed line presents the function to restrict the non-linear distortion α that can be introduced.

In FIG. 7, the results of the simulation are illustrated. Since the coherence level γ between the channels is high, non-linear distortion α is adaptively added to a maximum level of 0.5 except when there are talker position changes or background music. The result is a good misalignment performance with informal listening tests having shown that a better perceived quality of the background music sequence is achieved.

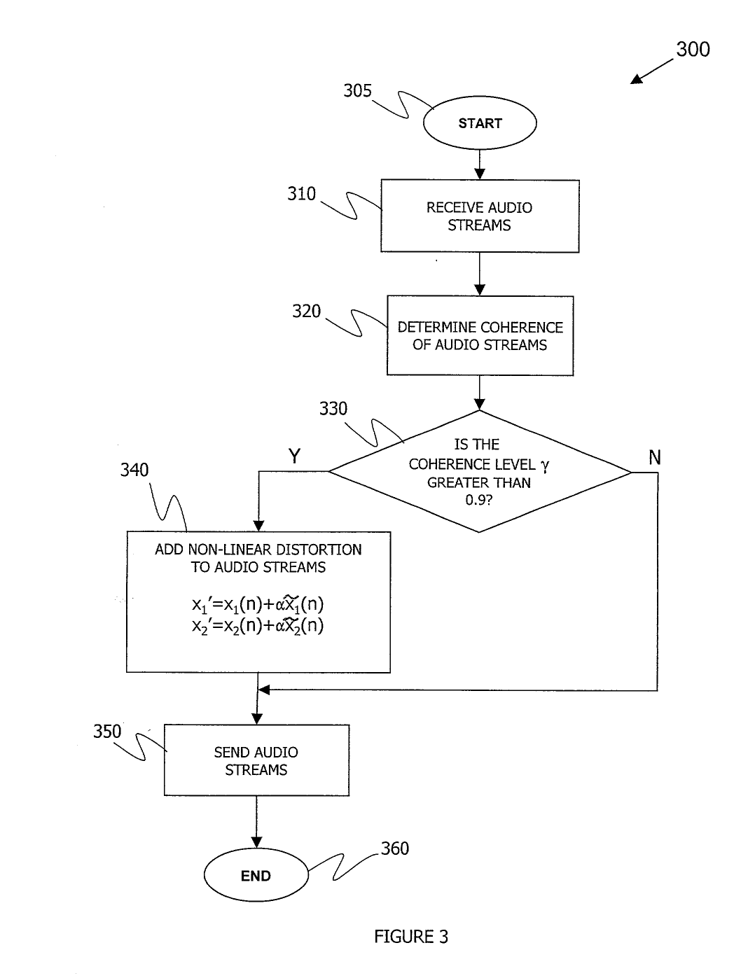

Turning now to FIG. 3, illustrated is a flow diagram of an embodiment of a method, generally designated 300, of managing distortion associated with an acoustic echo canceler, constructed in accordance with the principles of the present invention. The method 300 starts in a step 305 with an intent to manage distortion associated with an acoustic echo canceler.

After starting, a distortion manager receives audio streams in a step 310. In one embodiment, the distortion manager may receive the audio streams from a transmission room of a stereophonic teleconferencing system. The distortion manager may receive the audio streams via a conventional telecommunications network that may be either wireless, hardwired or a combination of the two.

A coherence ascertainer of the distortion manager then determines the coherence of the audio streams in a step 320. The coherence ascertainer may determine the coherence of the audio streams from Equation (27) where an estimated coherence level \hat{γ} is an estimate of the coherence level γ of the audio streams.

After determining the coherence, the coherence ascertainer determines if the coherence level γ is greater than 0.9 in a first decisional step 330. As discussed above with respect to the step 320, an estimate of the coherence level γ of the audio streams may be determined by the coherence ascertainer employing Equation (27).

If it is determined that the coherence level γ is greater than 0.9, then an adaptive distortion adder selectively adds non-linear distortion α to the audio streams in a step 340. In a preferred embodiment, the adaptive distortion adder selectively adds non-linear distortion α to the audio streams by employing a half-wave rectifier. In FIG. 3, the half-wave rectifier is represented in the step 340 by Equation (30) and Equation (31).

$$\begin{matrix}{x}_{1}^{′} ( n ) = {x}_{1} ( n ) + \frac{α}{2} [ {x}_{1} ( n ) + \vert {x}_{1} ( n ) \vert ] = {x}_{1} ( n ) + α {\tilde{x}}_{1} ( n ) . & ( 30 ) \\ {x}_{2}^{′} ( n ) = {x}_{2} ( n ) + \frac{α}{2} [ {x}_{2} ( n ) - \vert {x}_{2} ( n ) \vert ] = {x}_{2} ( n ) + α {\tilde{x}}_{2} ( n ) . & ( 31 )\end{matrix}$$

In some embodiments, non-linear distortion α is only added to one of the audio streams. In another embodiment, a maximum amount of non-linear distortion α may be added to at least one of the audio streams. In yet another embodiment, however, varying amounts of non-linear distortion α may be added to at least one of the audio streams.

After the non-linear distortion α is selectively added to the audio streams, the distortion manager then sends the processed audio streams to their destination in a step 350. Finally, the managing of distortion in an acoustic echo canceler ends in a step 360. Returning now to the first decisional step 330, if the coherence level γ is not greater than 0.9, the method 300 proceeds to the step 350 and continues as before.

Although the present invention has been described in detail, those skilled in the art should understand that they can make various changes, substitutions and alterations herein without departing from the spirit and scope of the invention in its broadest form.

What is claimed is:

1. A distortion manager for use with an acoustic echo canceler, comprising:
a coherence ascertainer configured to determine a coherency between audio streams; and
an adaptive distortion adder coupled to said coherence ascertainer configured to selectively add non-linear distortion to at least one of said audio streams based on said coherency.

2. The distortion manager as recited in claim 1 wherein said coherency ascertainer is configured to determine said coherency between more than two audio streams.

3. The distortion manager as recited in claim 1 wherein said non-linear distortion is produced by employing a half-wave rectifier.

4. The distortion manager as recited in claim 1 wherein a level of said non-linear distortion is a maximum of about 0.5.

5. The distortion manager as recited in claim 1 wherein said audio streams originate from a transmitting location of a stereophonic teleconferencing system.

6. The distortion manager as recited in claim 1 wherein said non-linear distortion is only added when a level of said coherency is equal to or is greater than about 0.9.

7. The distortion manager as recited in claim 1 wherein varying amounts of said non-linear distortion are added based on a level of said coherency.

8. A method of managing distortion associated with an acoustic echo canceler, comprising:
determining a coherence between audio streams; and
adding non-linear distortion selectively to at least one of said audio streams based on said coherence.

9. The method as recited in claim 8 wherein said determining said coherence is between more than two audio streams.

10. The method as recited in claim 8 further comprising producing said non-linear distortion by employing a half-wave rectifier.

11. The method as recited in claim 8 wherein a level of said non-linear distortion is a maximum of about 0.5.

12. The method as recited in claim 8 wherein said audio streams originate from a transmitting location of a stereophonic teleconferencing system.

13. The method as recited in claim 8 wherein said non-linear distortion is only added when a level of said coherency is equal to or greater than 0.9.

14. The method as recited in claim 8 further comprising adding varying amounts of said non-linear distortion based on a level of said coherency.

15. An acoustic echo canceler for a stereophonic teleconferencing system, comprising:
an echo estimator that produces a total echo estimate of individual echo paths in an receiving location by filtering audio streams from a transmitting location based on estimated impulse responses of said receiving location;
an echo error determiner that generates a signal representing the difference between said total echo estimate and a signal at said receiving location representing at least acoustic echo signals; and
a distortion manager, including:
a coherence ascertainer that determines a coherency between said audio streams; and
an adaptive distortion adder coupled to said coherence ascertainer that selectively adds non-linear distortion to at least one of said audio streams based on said coherency.

16. The acoustic echo canceler as recited in claim 15 wherein said coherency ascertainer is configured to determine said coherency between more than two audio streams.

17. The acoustic echo canceler as recited in claim 15 wherein said non-linear distortion is produced by a half-wave rectifier.

18. The acoustic echo canceler as recited in claim 15 wherein said non-linear distortion is a maximum of about 0.5.

19. The acoustic echo canceler as recited in claim 15 wherein said non-linear distortion is only added when a level of said coherency is equal to or greater than about 0.9.

20. The acoustic echo canceler as recited in claim 15 wherein varying amounts of said non-linear distortion are added based on a level of said coherency.
